(19) United States
(12) Patent Application Publication
(10) Pub. No.: US 2021/0145499 A1
(43) Pub. Date: May 20, 2021

(54) COLD PLASMA MEDICAL DEVICE

(71) Applicant: MURALI RAM, ADDISON, TX (US)

(72) Inventor: MURALI RAM, ADDISON, TX (US)

(21) Appl. No.: 17/100,749

(22) Filed: Nov. 20, 2020

Related U.S. Application Data
(60) Provisional application No. 62/938,147, filed on Nov. 20, 2019.

(51) Int. Cl. A61B 18/00

(52) U.S. Cl. CPC A61B 18/00; A61B 2018/00452; A61B 2018/00023; A61B 2018/00583

(57) ABSTRACT

A cold plasma medical device includes a plasma generator and a nozzle. The plasma generator is located within a housing. The plasma generator is configured to generate key RONs radicals from cold atmospheric plasma at energy levels less than 4 watts. The nozzle is in communication with the plasma generator and is configured to receive the cold atmospheric plasma and to enhance the RONs. The nozzle contains an electrode and a catalyst. The electrode is configured to converge the cold atmospheric plasma into the nozzle. The catalyst is adjacent the electrode and is configured to enhance the cold atmospheric plasma RONs. A controller is configured to regulate the performance of the medical device. In use, plasma with enhanced RONs is discharged through a tip of the nozzle.

### CROSS REFERENCE TO RELATED APPLICATIONS

This application claims the benefit of an earlier filing date and right of priority to U.S. Provisional Application No. 62/938,147, filed 20 Nov. 2020, the contents of which is incorporated by reference herein in its entirety.

### BACKGROUND OF THE INVENTION

### 1. Field of the Invention

The present application relates to a portable, homecare, cold plasma medical device with enhanced reactive species technology for disinfecting wounds in human cells.

### 2. Description of Related Art

Many companies have conducted research on Plasma usage in the medical field, with a focus on wound care treatment. Areas of focus have involved developing a cold plasma device for high-volume, high-intensity plasma. This is being used with clinic-based equipment outside of the equipment with close skin contact. Some focus has been directed to a portable unit designs to treat wound care thru cold plasma, again by focusing on high-volume. Research has been made toward atmospheric plasma systems on human cells, especially skin. A synergistic relationship between bacteria reduction (disinfection) and direct as well as indirect application of open air plasma systems has been discovered.

It has been found that by device nozzle design, plasma flow levels as well as the distance between plasma and the surface are critical success factors. It is seen that the industry focus is on generating high-volume and/or high-intensity plasma outside the unit by exposing it to atmospheric air. The challenges associated with the current industry trend has been highlighted before.

Although strides have been made in this area of technology within various industries, shortcomings remain. No patents for portable medical devices appear to have been approved in the US, leveraging plasma for consumer use at home to control wounds, primarily due to ionization and plasma stability impacts. Various theoretical research highlights the potential of plasma in the medical field, but the focus is on high-volume and high-intensity plasma. A goal of the present application is to design a safe device to overcome the industry challenges and obtain FDA as well as State approvals for controlling wound care at home. This will promote continuum quality care to patients at a competitive price.

### BRIEF SUMMARY OF THE INVENTION

It is an object of the present application to provide a safe medical treatment device to overcome industry challenges and obtain FDA as well as State approvals for controlling wound care at home. This will promote continuum quality care to patients at a competitive price. The medical device will generate enhanced RONs for selective application to a wound. The power used to generate the RONs is less than 4 watts.

It is a further object of the present application that the device be classified as a non-contact and chemical-free, painless, portable, and low power-consuming in-home medical device. In the alternative, it can also be classified under generation and enhancement of RONs radicals for successful disinfection of human cells.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 shows a breakdown of a cold plasma medical device in accordance with an embodiment of the present application.

FIG. 2 is an exemplary set up of the cold plasma medical device of FIG. 1.

FIGS. 3-7 are graphs providing an optical spectroscopic analysis of the cold plasma medical device of FIG. 1 at 2.3 watts (12-volts) and 1.5 watts (8-volts) input power using YAG crystal as a catalyst.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The embodiments and method of the present application are illustrated in the associated drawings. Upon familiarizing oneself with this technology the following should be understood: Plasma represents the fourth state of matter after solid, liquid, and gas. Plasma is further classified into hot, warm, cold and ultracold plasmas based on different characteristics. Entities present in plasma include positively and negatively charged ions, electrons, atoms, molecules, photons, elements of the ultraviolet and infrared light spectrum and localized electric fields. A magnetic field can also be incorporated to enhance the plasma. Plasma also spans a vast, parametric range of densities and temperatures. The transition between plasma types is often discontinuous and can jump suddenly at higher volumes, leading to instability and unsafe conditions. Plasma systems for biomedical applications involve the application of electrical energy to biologically inactive gases (such as air, argon, helium or mixtures thereof). In general, cold plasma devices can be categorized in two main types producing an ‘indirect’ or a ‘direct’ plasma for treatments. ‘Indirect’ plasma devices are more suitable for medical devices. The major portion of the applied electrical energy is used to produce high energy electrons responsible for generating reactive oxygen and nitrogen species (“RONs”). Since electrons have a very small thermal capacity, the temperature of the plasma remains low, which makes its application in biomedical research and medical devices possible.

Typically, the active hydroxyl (OH) and nitric oxide (NO) radicals with RONs penetrate through the cell wall and cause the redox reactions to deactivate the cell functions. Photo-catalytic technology can inactivate many microorganisms including bacteria such as E. coli, acidophilus, serratia domonasstutzeri, Bacillus pumilus, Streptococcus mutans, streptococcus, pseudomonas, fungi such as Candida albicans, yeasts such as cerevisiae, algae such as Chlorella vulgaris and viruses such as phage MS2, fragilis bacteriophage, Poliovirus, Cryptosporidium and Giardia intestinalis. Nitric Oxide (NO) is an important molecule which has been shown to regulate many processes in human skin physiology, influencing apoptotic pathways. Furthermore, it is involved in the regulation of proliferation and differentiation of human keratinocytes and promotes wound healing, disinfection, anti-inflammation, antimicrobial effects. It scavenges oxygen radicals as well as influencing microcirculation due to its effect as vasodilator. Thus, NO-generating plasma could be used to treat different skin and wound conditions.

Research in the field of disinfection by cold plasma for eradicating organic, inorganic and microbial pollutants has been well documented. The research also highlights that exposure to RONs at preferred levels for 30 to 60 seconds periodically under the guidance of a physician can inactivate many microorganisms highlighted above.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

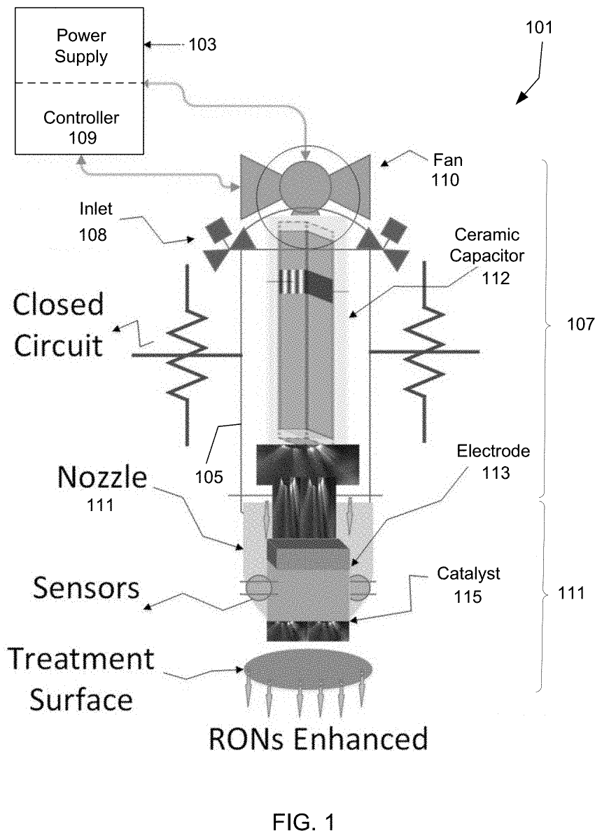

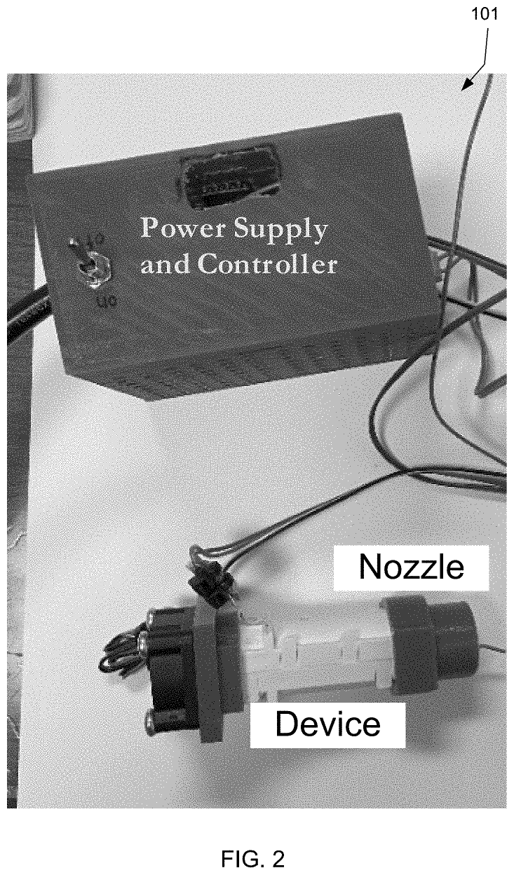

Referring now to FIGS. 1 and 2 in the drawings, an embodiment and exemplary perspective set up of a cold plasma device is illustrated. The present application involves a cold plasma medical device 101 operative within the universe of palliative wound care. The device 101 can be classified as a non-contact and chemical-free, painless, portable, and low power-consuming in-home medical device. In the alternative, it can also be classified under generation and enhancement of RONs radicals for successful disinfection of human cells. Device 101 includes a plasma generator 107 and a nozzle 111. The plasma generator 107 is located within a housing 105. The device 101 is regulated and operated via a power supply 103 and a controller 109. The nozzle 111 includes an electrode 113, and a catalyst 115.

One embodiment of the present application contemplates a battery-operated, non-contact, therapeutic portable medical device intended for home use to disinfect chronic wounds from key staph, strept, pseudomonas and propionic bacteria, as well as fungi like Candida albicans and viruses. The device of the present application is not herein limited to that shown in the Figures but is capable of being represented via various styles and features in line with the scope of the present disclosure.

It is noted that an embodiment of the medical device 101 disclosed generates key RONs radicals from cold atmospheric plasma at low levels inside the device as opposed to high levels/high intensity outside the device as seen with others. The RONs radicals are enhanced by integrating with a high-k dielectric material, but research on a photocatalyst with doping, graphene oxide and a plasmonic material is underway. The closed loop system not only simplifies design, but also maximizes patient safety. In an effort to increase safety further, cold plasma is generated at less than 4-watts to avoid skin burns. Lower input power is possible with a properly suited catalyst and nozzle circumference for optimum RONs enhancement and effective surface application. This portable device 101 will help wound care patients control wound infection, minimize hospital visits and promote palliative care by roughly spending a couple of minutes a day in the comfort of their home.

NO, NOx, O and O3, [amongst others], are [common] RONs that are generated in the form of ions at low levels from cold plasma by leveraging room air. Housing 105 includes inlets 108 and an optional fan 110 to facilitate air flow into the plasma generator 107. Cold plasma can be generated by charging a commercially available ceramic unit 112. When integrated with a photo catalyst such as titanium dioxide (TiO2) with doping and/or a surface plasmon-generating material such as silver (Ag), aluminum (Al) or magnesium (Mg), or graphene oxide or a high-k dielectric material such as yttrium aluminum garnet (YAG), the RONs are enhanced. Nitric Oxide (NO) enhancements can increase further during interaction with a moist surface, per research studies on bio-solutions. The size and structure of the photocatalyst, graphene oxide, high-k dielectric material and surface plasmon can be either nanoscale, such as nanoparticles or nanowires, or microscale, such as fine fibers or surfaces coated with thin films, both of which are activated by plasma sources. The intention is to choose a metal, alloy or refractory metal oxide based on their disinfecting and radical enhancing properties. This process of ion enhancement in a medical device not only makes it stable and safe for home use, but also provides recommended levels of RONs for natural disinfection from key staph, strept, pseudomonas, propionic bacteria, Candida albicans fungi and viruses present in common wounds.

This technology is packaged within a portable, battery-operated (or otherwise powered) medical device to allow patients the convenience of using such a device in their home for the purpose of disinfecting disease-borne bacterial, fungal, viral or other microbial cells. Although the device may be battery-operated, any power source 103 may be used, such as any AC or DC power source. When battery operated, one or two 6 to 9-volt batteries in series may be sufficient. The housing 105 of the device 101 may be formed in many different shapes but one shape conceived of is a pen-type shape to enhance portability. One skilled in the art will understand that the device could be powered in other ways as well.

It is understood that a goal of the cold plasma device 101 of the present application is to generate, energize and enhance the RONs inside a nozzle 111, thereby not only optimizing patient safety, but also reducing the levels and volume of plasma to safe and stable levels. Cold plasma can be automatically drawn towards the nozzle 111 by placing a conductive material like aluminum (Al) crystal in close proximity (graphene is under scrutiny) to initiate an electrical dis-charge. Converged cold plasma is then passed thru a catalyst 115 like high-k dielectric crystal YAG (or a photocatalyst with doping or a plasmonic material or graphene oxide or a plasmonic material) to achieve RONs enhancement. Confining the plasma within the nozzle 111 will reduce open plasma exposure to the wound, thereby avoiding burns.

A method of use of the device may vary depending on conditions of the patient and upon the recommendation by competent medical professional. In way of example, treating the wounds may last for 30-60 seconds, with a frequency of 1 to 2 times a day. Palliative wound care patients at home (per physician recommendations) may expose their wound(s) to the present portable plasma device for a specified amount of time for treatment at a prescribed frequency on a daily basis. The device may include a “poke-yoke” design to disconnect the power supply at the maximum threshold limit. It is desired that the device provide patients the flexibility to select one of the preset levels as required per additional recommendations. The same design feature will be incorporated to control the frequency of treatment.

Ideally, patients should hold the device slightly above the wound to cover the area without touching the wound directly and turn on a power switch. To maintain hygiene, the patient should clean the device nozzle tip with a sterile wiping cloth before and after use. The patient should clean the wound with an antiseptic (per physician's advise) if the wound is dry as a moist surface is required for plasma generation. The generation of RONs and the associated enhancement is self-contained inside the nozzle of the device to maintain safety. The unit will automatically switch off upon reaching the set threshold timer and/or through a feedback loop from a sensor. The patient then will repeat this process for the prescribed frequency or until instructed otherwise by a physician.

Such treatment can likely reduce the number of hospital visits by wounded patients. Per numerous healthcare research studies, development of home-care devices can not only reduce overall healthcare costs but also boost patient lifestyle by virtually eliminating the need to be in a hospital setting. There are also numerous benefits for soldiers in remote locations who get hurt in combat. As an easy to transport device, wounds can be controlled and disinfected in a timely manner, minimizing the need for advanced personnel as well as preventing any chronic conditions. Other steps in the treatment process may include cleaning the wounds using standard methods, such that it is free of any grease, ointments or lotions.

The cold plasma medical device of the present application includes many features and advantages. Some of which are provided below:
The ability to leverage low volume cold plasma using atmospheric air for disinfection of harmful microbial cells that inhibit the healing of typical wounds.
Directing the photons from the plasma sources onto a high-k dielectric material such as yttrium aluminum garnet (YAG), or a photocatalyst material such as titanium dioxide (TiO2) with doping, or a plasmonic materials such as silver (Ag), or graphene oxide as a catalyst to enhance RONs.
Novel design methods such as the size and structure of the catalyst in nanoscale (nanoparticles or nanowires) or microscale (fine fibers) forms that are activated by photon producing sources, both of which are capable of absorbing photons and provide electrons or holes required to enhance the RONs under room air conditions.
Spectrometric analysis of plasma to gauge RON enhancements.
Minimal photon exposure to the skin, achieved via special nozzle designs in order to confine most of the photons from the sources onto the RONs enhancing materials.
Embedded timing device directly on the power supply board, as a part of the overall “poke-yoke” design.
Lower volume of cold plasma requirement and graphene oxide, resulting from more focus on enhancement of RONs via high-k dielectric materials, photocatalysts, or surface plasmons for disinfection in human cells.
Enhancing the RONs in cold plasma inside the nozzle, thus increasing the stability and safety of the device usage.
Activating the plasma generation only when the device is in close proximity to wounded area even when the unit is turned on due to its conductive nature.
Design of two-part portable, in-home, touch-free and painless device.
Innovative combination of technologies for disinfection for various healthcare applications, such as human cells.

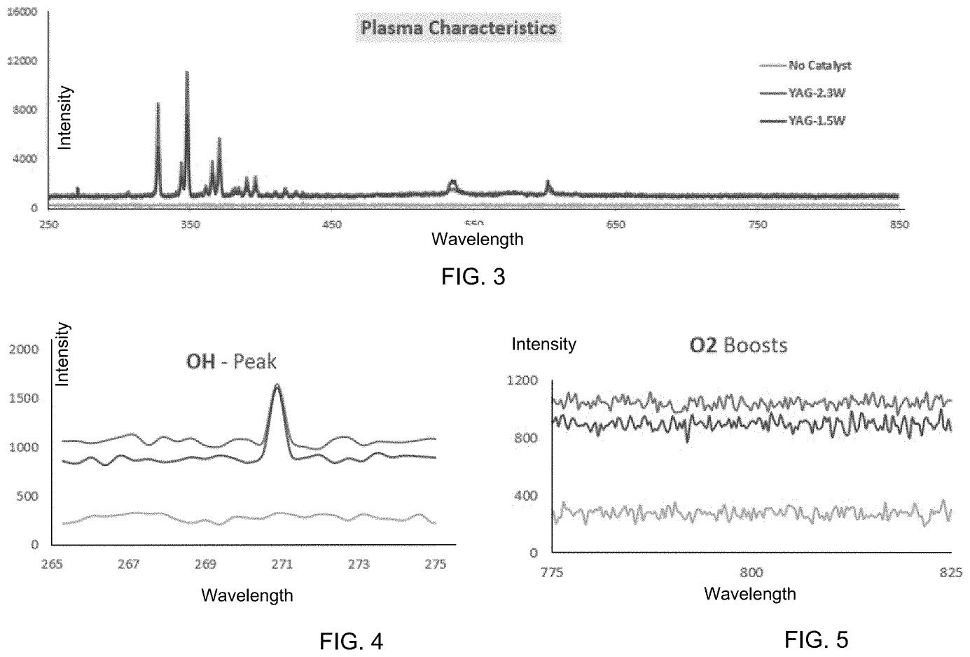

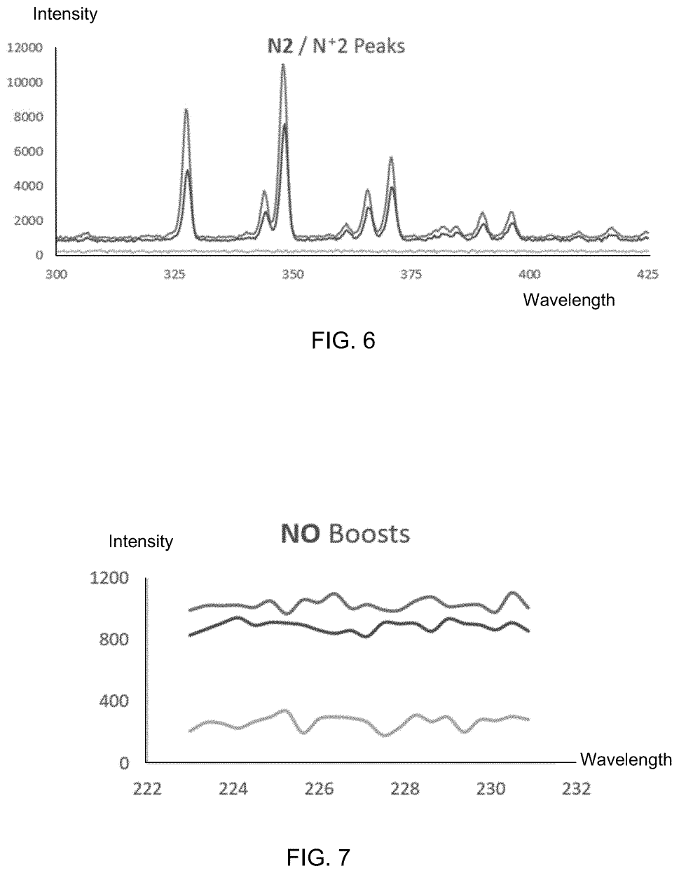

Referring now also to FIGS. 3-7 in the drawings, graphs showing the effects of power differences on the intensity of the plasma is illustrated. It clearly indicates that there are enhancements due to excited nitrogen, oxygen, nitric oxide and hydroxyl radicals compared to the baseline. It also shows that the input power is partially correlated to plasma intensity, warranting more scrutiny to find an optimum level. It also means that treatment times can be potentially reduced with higher reactive species exposure thus avoiding thermal effects on the surface. More analysis is underway with other dielectric, photocatalytic, graphene oxide and plasmonic materials as catalysts.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A cold plasma medical device, comprising:
a plasma generator located within a housing, the plasma generator configured to generate key RONs radicals from cold atmospheric plasma at energy levels less than 4 watts;
a nozzle in communication with the plasma generator and configured to receive the cold atmospheric plasma and to enhance the RONs, the nozzle containing:
an electrode configured to converge the cold atmospheric plasma into the nozzle; and
a catalyst adjacent the electrode and configured to enhance the cold atmospheric plasma RONs; and
a controller configured to regulate the performance of the medical device;
wherein plasma is discharged through a tip of the nozzle.

2. The device of claim 1, wherein the catalyst is a high-k dielectric material.

3. The device of claim 1, wherein the catalyst is a photocatalytic material.

4. The device of claim 1, wherein the catalyst is a plasmonic material.

5. The device of claim 1, wherein the electrode is made of aluminum.

6. The device of claim 1, wherein the electrode is made of graphene.

7. The device of claim 1, wherein the nozzle is interchangeable to permit adjustment of at least one of a catalyst and nozzle circumference.

8. The device of claim 1, further comprising:
a fan to force air flow through the housing.

9. The device of claim 1, further comprising:
a power supply electrically coupled to the plasma generator.

10. The device of claim 9, wherein the power supply is rechargeable.

11. The device of claim 9, wherein the power supply is a battery.

12. A method of treating a wound, comprising:
obtaining a cold plasma medical device of claim 1;
activating the cold plasma medical device;
selecting the nozzle and coupling it to the plasma generator; and
holding the cold plasma medical device off the wound.

13. The device of claim 12, further comprising:
interchanging the nozzle to adjust the size of the nozzle tip and a catalyst.

14. The device of claim 12, further comprising:
setting a timer to regulate the duration of operation of the cold plasma medical device.
